(19) United States
(12) Patent Application Publication
(10) Pub. No.: US 2015/0265299 A1
(43) Pub. Date: Sep. 24, 2015

(54) Minimally Invasive Thrombectomy

(71) Applicants: Christopher J. COOPER, (US); Mohammad H. ELAHINIA, (US); Andrew J. PUFFER, (US); Michael J. KOLUDROVICH, (US); THE UNIVERSITY OF TOLEDO, Toledo, OH (US)

(72) Inventors: Christopher J. Cooper, Perrysburg, OH (US); Mohammad H. Elahinia, Sylvania, OH (US); Michael J. Koludrovich, North Royalton, OH (US); Andrew J. Puffer, Toledo, OH (US)

(21) Appl. No.: 14/433,278

(22) PCT Filed: Oct. 2, 2013
(86) PCT No.: PCT/US13/62998

§ 371 Date: Apr. 2, 2015

Related U.S. Application Data
(60) Provisional application No. 61/709,202, filed on Oct. 3, 2012.

(51) Int. Cl. A61B 17/221

(52) U.S. Cl. CPC A61B 17/221; A61B 2017/22038

(57) ABSTRACT

A minimally invasive blood clot capturing invention made of nitinol. The nitinol is shaped into a plurality of fingers to form a frame for a basket and funnel to capture and remove blood clots. The basket and funnel being delivered to the blood clot by a catheter. The basket and funnel are capable of being collapsed within a catheter, capable of being deployed into a blood vessel, and capable of being retracted into the catheter for removal from the blood vessel.

### CROSS REFERENCE TO RELATED APPLICATIONS

This application claims the benefit of U.S. provisional patent application Ser. No. 61/709,202 filed October 3, 2012 and PCT/U513/62998 filed on Oct. 2, 2013.

### STATEMENT REGARDING FEDERALLY SPONSORED RESEARCH

The invention was not made with any government support and the government has no rights in the invention.

### BACKGROUND OF THE INVENTION

Many vascular system problems stem from insufficient blood flow to the heart. One of the main causes is a blockage within veins known as a blood clot, or thrombus. This can occur after trauma, surgery, or other phenomenological reasons. Clinical data indicates that clot removal inventions and procedures can reduce the need for an amputation by 80 percent. The ultimate goal of any modality to treat these conditions of the arterial or venous system is to remove the blockage or restore patency, quickly, safely, and cost effectively. This can be achieved by thrombus dissolution, fragmentation, thrombus aspiration or a combination of these methods.

Percutaneous thrombectomy refers to the removal of thrombus using non-surgical methods. Percutaneous thrombectomy can be used to remove thrombus from arteries, veins and vascular grafts and can be used alone, as a primary procedure, or in combination with transcatheter thrombolysis or angioplasty and stenting.

Catheter directed thrombectomy and thrombolysis is less traumatic and avoids the morbidity and mortality associated with conventional surgical techniques. It also has the advantage of providing diagnostic information about associated vascular diseases and to treat coexisting lesions. As a result, there has been a push for the use of percutaneous mechanical thrombectomy (PMT) devices. These devices offer a key advantage over surgical thrombectomy or thrombolysis. The concept of the mechanical thrombectomy is attractive, however, developing a miniature device that can quickly and atraumatically restore patency to a vessel without creating some degree of distal embolization is a goal that still eludes the medical community.

An invention based on the use of super elastic alloy nitinol has been developed that offers several advantages over PMT device currently on the market. This invention provides a higher degree of authority and maneuverability for capturing and removing blood clots. A major issue with existing inventions is shearing off of smaller particles (embolic particles) during the process of clot removal. Thromboembolism occurs when either the clot itself or the embolic particles travel downstream and occlude another vessel in the body, known as a secondary clot. Secondary clots are known to lead to fatal conditions such as pulmonary embolism, when the secondary clots move to pulmonary arteries in the lunges, or a stroke, when the secondary clots occlude the vessels in the brain. In the the number of fatalities due to pulmonary embolism alone is known to be approximately 200,000 per year. The invention is capable of removing a thrombus while minimizing embolic particles and therefore reducing the risk of secondary clot formation and the related complications and fatalities.

### SUMMARY OF THE INVENTION

The present invention generally refers to a minimally invasive blood clot capturing device made of nitinol. The invention is deployed by a catheter that is introduced into the body using the modified Seldinger technique. The catheter is driven to a blood clot and the operator deploys the invention out of the catheter. The invention evacuates pieces of thrombus without shearing the blood clot into smaller pieces. Current PMT devices that operate to pull-back and capture a thrombus have no way of guiding a blood clot into an exiting catheter without shearing pieces of blood clot which can escape downstream and create, in some instances, problems that are worse than the initial obstruction. In most cases, the blood clot has a diameter that is much larger than that of the exiting catheter. With the introduction of this invention, loss of emboli is drastically reduced in most situations.

Various aspects of this invention will become apparent to those skilled in the art from the following detailed description of the preferred embodiments, when read in light of the accompanying drawings.

### BRIEF DESCRIPTION OF THE DRAWINGS

FIG. 1 is a perspective view of the laser spiral cut nitinol tube prior to shape setting.

FIG. 2 is a perspective view of the laser spiral cut nitinol tube after shape setting.

FIG. 3 is a perspective view of the invention, showing both elements of the invention, the basket and funnel, deployed to capture a blood clot between the two elements.

FIG. 4 is an expanded perspective view of the collection basket.

FIG. 5 is an expanded perspective view of the funnel.

FIG. 6 is a perspective view of the invention.

FIG. 7 is a schematic view of FIG. 3.

FIG. 8 is a graph showing the results of testing, error bars show ±standard deviation.

FIG. 9 is a perspective view of the laser straight cut nitinol tube prior to shape setting.

FIG. 10 is a perspective view of the laser straight cut nitinol tube after shape setting.

### DETAILED DESCRIPTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

Before the instant invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. Instead, the scope of the present invention will be established by the appended claims.

It must be noted that, as used in this specification and the appended claims, the singular forms “a,” “an” and “the” include plural reference unless the context clearly dictates otherwise. Unless defined otherwise all technical and scientific terms used herein have the same meaning as commonly understood to one of ordinary skill in the art to which this invention belongs.

Where a range of values is provided, it is understood that each intervening value, to the tenth of the unit of the lower limit, unless the context clearly dictates otherwise, between the upper and lower limit of that range and any other stated or intervening value in that stated range, is encompassed within the invention. The upper and lower limits of these smaller ranges may independently be included in the smaller ranges, and such embodiments are also encompassed within the invention, subject to any specifically excluded limit in the stated range. Where the stated range includes one or both of the limits, ranges excluding either or both of those included limits are also included in the invention.

Throughout the entire specification, including the claims, the word “comprise” and variations of the word, such as “comprising” and “comprises,” as well as “have,” having,” “includes,” “include,” and “including,” and variations thereof, means that the named steps, elements or materials to which it refers are essential, but other steps, elements, or materials may be added and still form a construct with the scope of the claim or disclosure. When recited in describing the invention and in a claim, it means that the invention and what is claimed is considered to what follows and potentially more. These terms, particularly when applied to claims, are inclusive or open-ended and do not exclude additional, unrecited elements or methods steps.

The term “nitinol” herein is used to describe a metal alloy comprised of nickel and titanium where the two elements are present in approximately equiatomic percentages. The term super elastic herein is used to describe a property of nitinol of a certain chemical composition in which a deformation is recovered without it being necessary to heat the nitinol alloy.

Thrombus is used to describe a blood clot, the final product of a blood coagulation step in hemostasis. As such the terms thrombus and clots are used interchangeably.

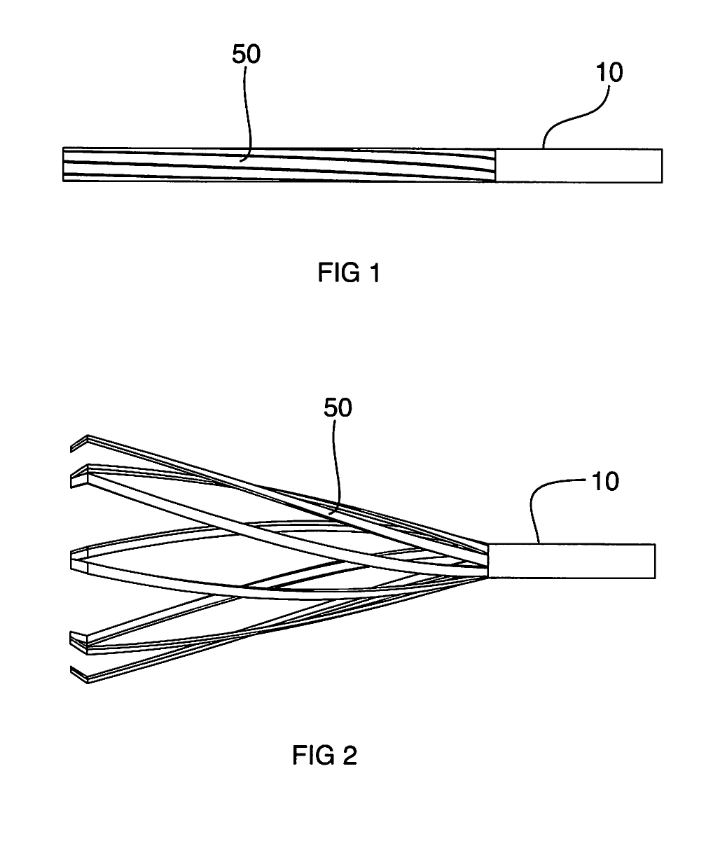

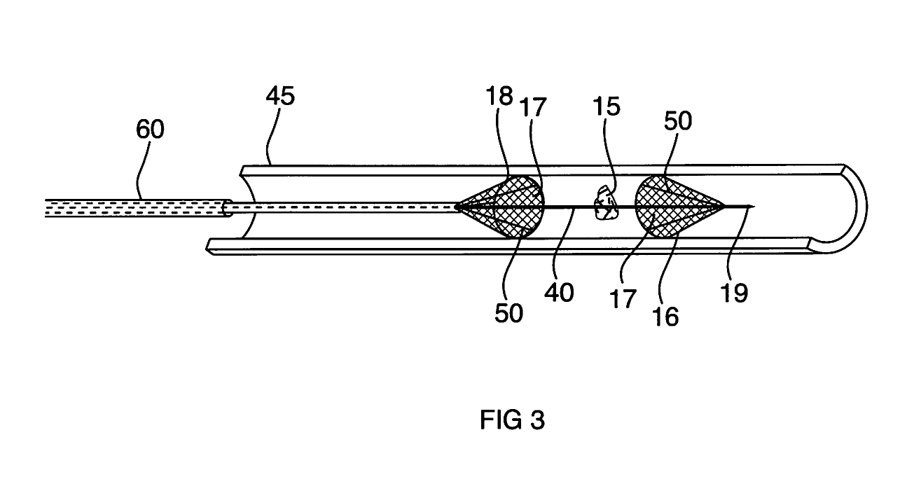

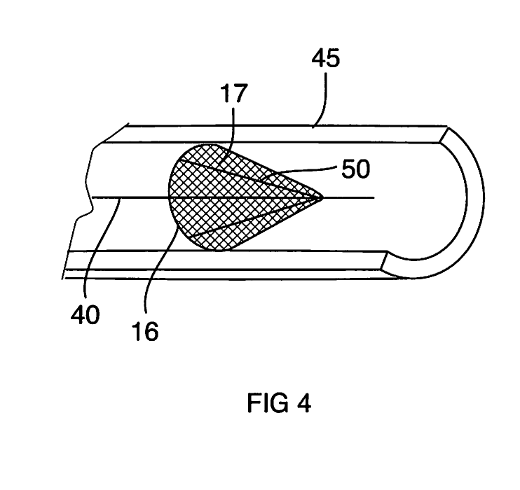

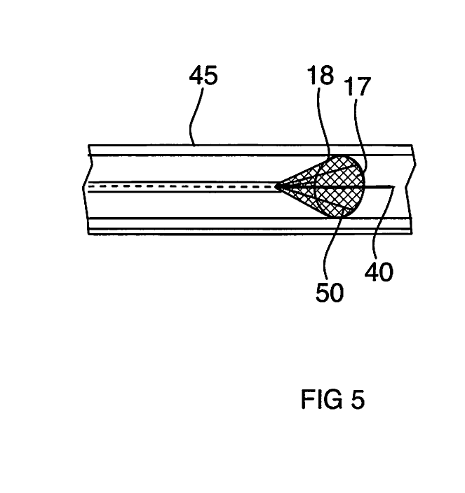

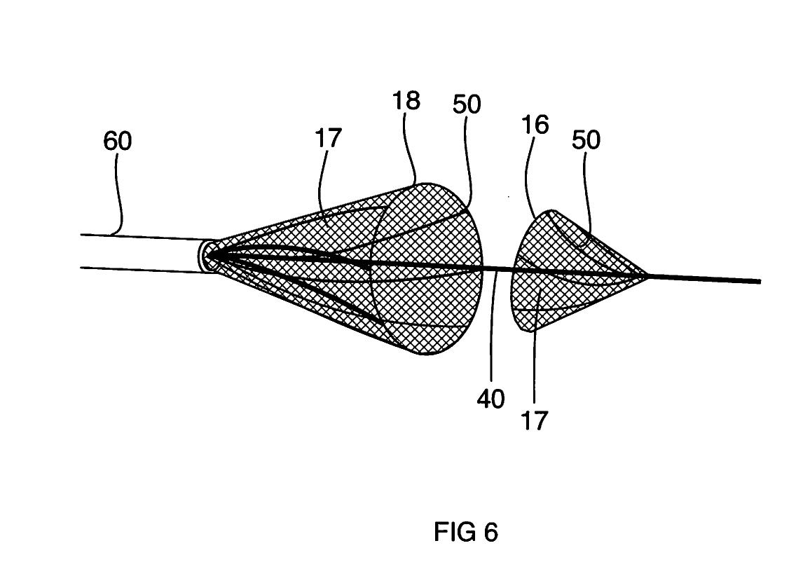

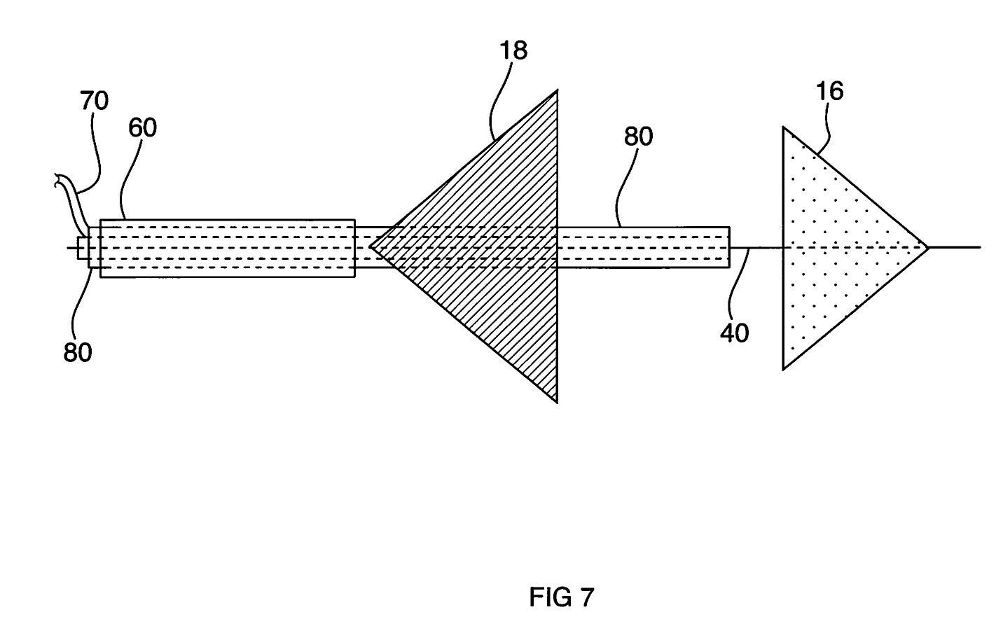

Referring now to FIGS. 1-3 the invention is a percutaneous catheter based device that uses a pair of super elastic nitinol capturing elements to aid in the collection of a thrombus and minimize distal embolization. The invention, being made of nitinol and once it is unrestricted by the catheter, changes shape to create a basket 16. The invention has two components of a super elastic nitinol tube 10 that have been partially laser cut longitudinally and shape-set to create a cone of super elastic fingers. FIG. 1 shows the nitinol tube 10 as cut by the laser cutting process prior to shape setting. This same cutting process, illustrated in FIG. 1, is used to create both, the basket 16 and a funnel 18. FIG. 2 illustrates how a plurality of fingers 50 are shape set into the expanded position. FIGS. 11-12 illustrate the preferred embodiment wherein the plurality of fingers 50 of the basket 16 and the funnel 18 can be spiraled or have a helix shape to maximize contact the nitinol fingers of the basket 16 and funnel 18. To further ensure contact of the nitinol fingers, the plurality of fingers 50 are spiraled in opposite directions. The basket 16 and the funnel 18 are covered in a blood permeable membrane 17 such as expanded Polytetrafluoroethylene (ePTFE), BioWeb, or other membranes, capable of allowing blood flow while capturing blood clots. To reduce the risk of puncturing a blood vessel 45, the plurality of fingers 50 may be rounded, further, the tips of the fingers 50 may be flattened and/or curved inward 51 as illustrated in FIG. 2. The basket 16 and the funnel 18 may be made from super elastic nitinol or other metal alloy exhibiting super elastic properties. The spiral orientation of the plurality of fingers 50 also reduces the risk of tearing the membrane 17.

In the preferred embodiment the invention is deployed using an outer catheter 60 and an inner catheter 80. The invention is deployed by constricting the basket 16 within n the inner catheter 80 and by constricting the funnel 18 with the outer catheter 60. The outer catheter is directed to the blood clot 15. The outer catheter is partially retracted to deploy and expand the funnel 18. The inner catheter 80 and the basket 16 are guided to the distal end of the blood clot 15 and the inner catheter is fully removed to deploy and expand the basket 16. A guide wire 40 is used to draw the basket 16 to the blood clot 15. Pulling the basket 16 through the blood clot 15 will cause the blood clot to lodge into the basket 16. A guide wire 40 is withdrawn to guide the basket 16 into the funnel 18. The funnel 18 and the basket 16 are drawn into the outer catheter 60 collapsing the funnel 18 onto the basket 16 and thus collapsing the basket inside the funnel and trapping the blood clot within. In other embodiments additional catheters 70 may be used to provide suction, deploy multiple baskets, or other devises to dislodge the blood clot 15.

In the preferred embodiment the funnel 18 and the basket 16 are used in conjunction, however it is envisioned that the funnel or basket could be deployed individually, or in combination with other PMT devices.

The basket 16 may have a probe 19 located on the end to assist in moving the basket to the distal end of the blood clot 15. The catheter can be withdrawn leaving behind and deploying the basket 16. Once the catheter is withdrawn the plurality of fingers 30 of the basket 16 expand to the predetermined shape. A similar process takes place proximal to the blood clot 15. The funnel 18 is advanced out of a constraining catheter where it expands to nearly the diameter of the vessel lumen. The super elastic property of nitinol assists in expanding the nitinol material that forms the basket 16 and the funnel 18. The guide wire 40 extends to the basket 16 and through the funnel 18. The guide wire 40 is advanced in a direction towards the funnel moving the basket towards the funnel. This movement brings the basket 16 into contact with the blood clot 15 whereby the basket can remove the blood clot from the blood vessel by trapping the blood clot within the basket 16.

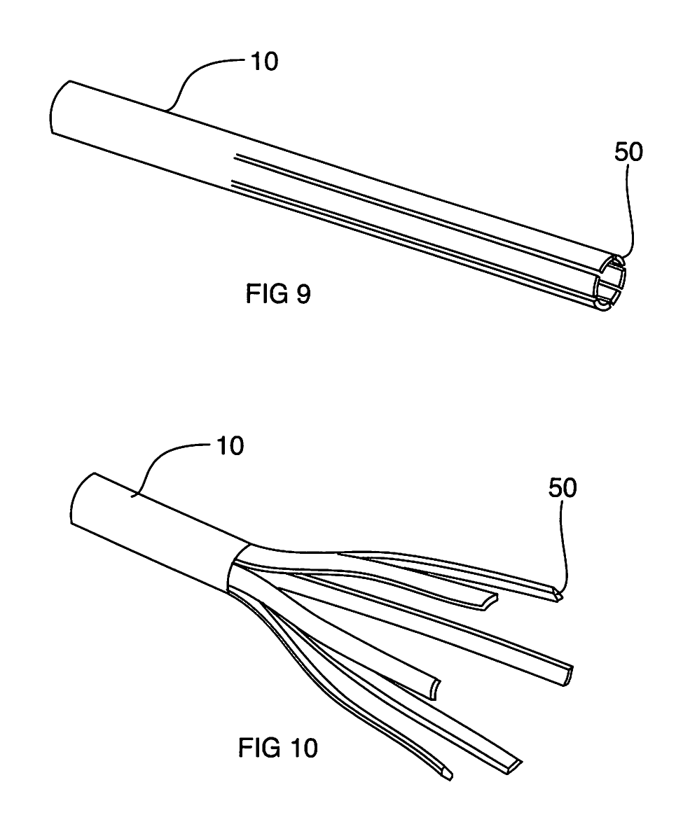

FIGS. 9 and 10 illustrate different embodiments of the invention wherein the plurality of fingers 50, are straight and not spiral shaped. Other shapes may be used for the plurality of fingers 50.

In sonic applications it may be preferable to have control wires that extend through the funnel 18 and engage the outer periphery of the basket 16. The control wires can be moved individually or as a group to position the outer periphery in a position adjacent to the clot 15. The control wires can help to position the basket 16 in the best position to capture the clot. In some instances the control wires can be used to assist the basket in removing the clot from the wall of the blood vessel.

A source of suction may be directed to the portion of the blood vessel that is located between the basket 16 and the funnel 18. The suction is used to remove fluid and particles from the blood vessel during the time that the blood clot 15 is being removed from the blood vessel. The basket is withdrawn capturing the blood clot 15. The basket 16 can be withdrawn and nested in the funnel 18. Aspiration of the blood clot 15 can be performed if desired by applying manual suction with a syringe on the funnel 18 catheter. Once nested, the two nitinol components of the invention collapse by withdrawing the funnel 18 catheter within the outer catheter 60 taking the basket 16 and clot with it.

In some applications the funnel 18 and the basket 16 can be positioned using separate catheters or the same catheter. Suction can be provided through a catheter that positions the basket 16, the funnel 18, or a separate catheter 70 may be used to provide the suction. It should also be appreciated that either the basket 16 or the funnel 18 may be deployed or opened first in the blood vessel depending on how the blood clot 15 is to be removed.

The basket 16 and the funnel 18 are made of a material which is opaque for medical imaging equipment to allow for monitoring of the placement of the basket and funnel relative to the blood clot 15. In some procedures it may be desirable to use a balloon on the distal or proximal side of the blood clot. The balloon can be used to stop or control the flow of blood through the vessel during the clot removal procedure.

Two components of super elastic nitinol tube 10 have been partially laser cut longitudinally and shape set to create a cone of super elastic fingers FIG. 2. A Techne FB-08 fluidized alumina furnace may be used for the shape setting.

Different embodiments of the invention can include varying diameters of nitinol tubing 10 as well as varying diameter of the final shape set size of collection basket 16 and/or funnel 18. Since the human circulatory system includes vessels of many different sizes, the invention may be scalable to be used in various locations of the body.

### EXAMPLE

A simulated circulatory system was built to test the present invention along with a commercially available PMT device. The setup features a reservoir of physiologic saline solution pumped through a system of tubing using a peristaltic pump. The network oftubes splits into a testing branch and a bypass branch. The testing branch has an acrylic chamber that is tapered to simulate an arterial or venous stenosis. This section of the testing environment was designed such that an artificial clot would become stuck in this section and the inventions could be used as they would be clinically. A three stage cascading filtration system was installed downstream from the testing chamber to capture any embolic particles.

A peristaltic pump (Ismatec MCP Standard) was programmed to simulate the pulsate flow from the heart. The maximum pressure was approximately 120 mmHg, the minimum pressure was approximately 80 mmHg, and the maximum velocity was about

$$3.5 \frac{m}{s} .$$

A 10 mL sample of fresh blood was transferred to an intermediate 15 mL test tube. A pipette was then used to transfer 9 mL of blood to twelve, 2 mL test tubes creating twelve samples of 7504 of blood. These twelve samples were allowed to incubate at 22° C. for 24 hours. This procedure was done twice: once to create twelve clots to test the present invention and once create twelve clots to test a commercially available PMT device.

A DiverCE Rapid Exchange Clot Extraction Catheter (INVATEC S.p.A. Roncadelle (Bs) Italy) was chosen as the commercially available PMT to test under the same conditions as the present invention. The DiverCE is an aspiration invention that uses manual suction with a syringe to evacuate a clot. There are two version of the DiverCE, one for “organized thrombus” and one for “fresh thrombus”. The version for fresh thrombus was used for this test.

Blood Clots were created and placed in the system. The system was then sealed and the pump was activated. The present invention was introduced via percutaneous puncture of the laboratory tubing. The invention was withdrawn and the blood clot 15 was captured and removed. This procedure was repeated twelve times. The DiverCE catheter was operated as outlined by the manufacturer's instructions for use in each of the twelve trials.

Data was collected using the three stage cascading filtration system. Stainless steel filters were used to capture any embolic material. Filter #1 closest to the clot had a pore opening size of 102 μm. Filter #2, the middle filter, had a pore opening size of 25 μm. The last filter, Filter #3, had a pore size of 5 μm. A set of three filters (#1, #2, and #3) were weighed prior to performing a capturing trial with either the present invention or the DiverCE. The filters were installed into the designed flanges and the test was conducted. After the capturing procedure was completed and the pump deactivated, the tubing was evacuated of saline via a laboratory vacuum such that all possible particles would be captured by the filtration system. The filters were removed, and allowed to dry for 24 hours. The filters were reweighed and any mass gain was recorded. New filters were used for each trial.

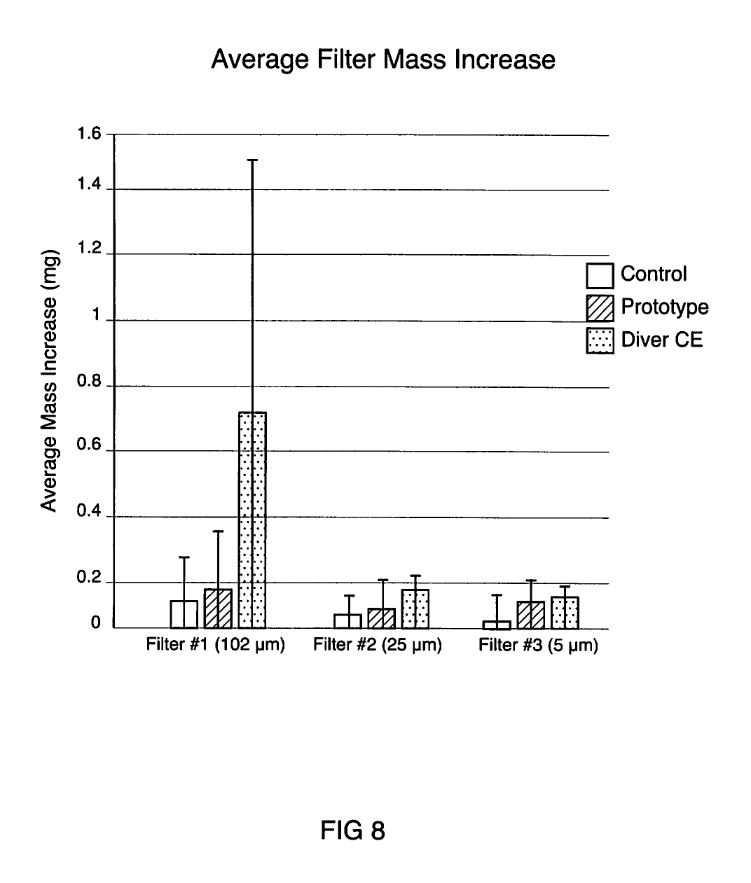

A control study was also conducted. Saline solution was allowed to flow over a series of three filters (#1, #2, and #3) for a period of twenty seconds. Filters were weighed prior to saline flow and after 24 hours of drying time. Any mass increase was recorded and results are shown in FIG. 8. As illustrated by FIG. 8, the present invention outperformed the commercially available DiverCE at each stage and drastically reduced embolic particles.

The principle and mode of operation of this invention have been explained and illustrated in its preferred embodiments. However, it must be understood that this invention may be practiced otherwise than as specifically explained and illustrated without departing from its spirit or scope.

What is claimed is:

1. Apparatus for conducting a thrombectomy in a blood vessel of a patient comprising:
an expandable basket disposed for positioning in the blood vessel on one side of a thrombus, the expandable basket being designed to remove the thrombus;
an expandable funnel positioned in adjacent spaced apart relationship to the expandable basket, the expandable funnel being disposed to collect embolic particles released as part of the removal of the thrombus; and
a guide extending to the expandable basket, the guide being positioned for advancing the expandable basket in a direction towards the expandable funnel to remove the thrombus.

2. The apparatus of claim 1 wherein the basket and funnel have a plurality of fingers that are made from a super elastic material.

3. The apparatus of claim 2 wherein the super-elastic material comprises Nitinol, a nickel titanium alloy.

4. The apparatus of claim 2 wherein a portion of an outer surface of the plurality of fingers of the basket and funnel are covered by a blood permeable material.

5. The apparatus of claim 4 wherein the blood permeable material is a biocompatible material.

6. The apparatus of claim 5 wherein the biocompatible material is expanded Polytetrafluoroethylene or Bioweb.

7. The apparatus of claim 4 wherein the apparatus has a deployed state, wherein the plurality of fingers of the basket and funnel expand and engage an interior wall of the blood vessel, and a delivery state wherein the plurality of fingers have a contracted configuration to allow insertion in the blood vessel within a delivery sheath.

8. The apparatus of claim 7 wherein the basket is designed to fit into the funnel, the funnel being designed to collapse around the basket and to collapse the basket whereby the thrombus is retained in the collapsed funnel and basket.

9. The apparatus of claim 7 wherein the delivery sheath is provided for positioning the basket, funnel and flexible guide wire in the blood vessel.

10. The apparatus of claim 4 wherein the plurality of fingers are disposed to increase contact between the funnel fingers and the basket fingers.

11. The apparatus of claim 10 wherein the plurality of fingers are disposed in a spiral orientation.

12. The apparatus of claim 4 wherein the plurality of fingers have a distal end that form the outer periphery of the basket and the funnel.

13. The apparatus of claim 12 wherein the distal ends of the fingers are displaced in a direction towards the interior of the basket and funnel.

14. The apparatus of claim 7 wherein the plurality of fingers are disposed in an opposing spiral orientation and the plurality of fingers of the basket and funnel overlap when the basket is in engagement with the funnel whereby the blood permeable material is protected from being punctured by the plurality of fingers.

15. A containment device for removing a blood clot of a patient comprising:
a tube made of a super elastic material;
a plurality of fingers formed in one end of the tube, the fingers having a proximal end adjacent the tube and a distal end spaced apart from the tube, the plurality of fingers being capable of expanding to form a frame for a device to remove blood clots.

16. The apparatus of claim 15 wherein the distal ends of the fingers are displaced in a direction towards the interior of the expanded frame.

17. The apparatus of claim 16 wherein the plurality of fingers are covered with a blood permeable material that has a porosity that will retain a blood clot.

18. The apparatus of claim 17 wherein a second containment device is disposed adjacent the first containment device, the first containment device being designed to fit within the second containment device to capture the blood clot.

19. The apparatus of claim 18 wherein the second containment device engages and collapses the first containment device around the blood clot.

20. The apparatus of claim 15 wherein the plurality of fingers disposed in a spiral orientation.
